(19) United States
(12) Patent Application Publication
(10) Pub. No.: US 2003/0225355 A1
(43) Pub. Date: Dec. 4, 2003

(54) Composite material for wound repair

(76) Inventor: Charles E. Butler, Houston, TX (US)

Correspondence Address: VINSON & ELKINS, L.L.P., 1001 FANNIN STREET, 2300 FIRST CITY TOWER, HOUSTON, TX, 77002-6760, US

(21) Appl. No.: 10/406,153

(22) Filed: Apr. 1, 2003

Related U.S. Application Data
(63) Continuation-in-part of application No. 09/164,481, filed on Oct. 1, 1998.
(60) Provisional application No. 60/369,063, filed on Apr. 1, 2002.

(51) Int. Cl. A61F 13/00; A61F 15/00

(52) U.S. Cl. 602/48

(57) ABSTRACT

A composite comprising a barrier material and a support material used for wound or tissue repair. Benefits include decreased adhesion to organs or other structures adjacent to the repair site, limited fluid flux, increased vascularization and cellular infiltration, decreased inflammation and reduced scar tissue formation.

### CROSS-REFERENCE TO RELATED APPLICATIONS

The present application claims the benefit of provisional U.S. Serial No. 60/369,063 filed Apr. 1, 2002.

### FIELD OF THE INVENTION

The present invention relates to compositions and methods for wound and tissue repair. More specifically, the present invention provides a composition including a support material and a barrier material, as well as methods for using the composition to facilitate wound and tissue repair.

### STATEMENT REGARDING FEDERALLY SPONSORED RESEARCH OR DEVELOPMENT

NA

### BACKGROUND OF THE INVENTION

A wide variety of implantable biomaterials has been used to repair tissue defects and tissue loss in mammals. Currently, such tissue repairs can only be done with prosthetic material or use of a section of autologous tissue from another location with similar functional characteristics, often from a different organ system. The use of prosthetic material is limited by its non-viability, lack of specialized function, immunologic reaction or rejection and increased risk of infection. Autologous tissue from a separate location is often used to replace tissue defects. For example, intestine can be used for esophageal replacement and bladder reconstruction, and urinary conduit can be used for ureter loss or bile duct replacement. Also, donor veins are used to replace arteries. Using autologous tissue for replacement requires a surgical procedure and tissue loss from an uninjured organ. In addition, the donor tissue often does not have the identical structural or function characteristics of the native tissue and suffers from lack of specific anatomic and physiologic function.

Prosthetic mesh, such as polypropylene, has historically been used as a support structure for such wound and tissue repair. Unfortunately, however, when using prosthetic mesh, adhesions form between intraperitoneal structures, such as bowel and omentum, and the repair site. Additionally, the repair site often exhibits irregular or inadequate cellular infiltration and neovascularization, resulting in excessive scarring and a thin tissue layer that is more susceptible to infection or other additional damage. Additionally, wound cavities are often created by raising soft tissue flaps which, after closure, lie directly adjacent to the support material. These wound cavities leak serous fluid and ooze blood which leads to seroma and hematoma formation. Formation of adhesions and inadequate infiltration and vascularization are associated with significant morbidity, resulting in, among other complications, obstruction, pain, female infertility, entercutaneous and other fistulas, seroma, wound infection, mesh extrusion, and bowel perforation. As a result, re-operative abdominal surgery is frequently required to repair the complications resulting from the adhesions.

Five fundamental strategies have been used to reduce abdominal adhesions and increase normal tissue formation: (1) limit peritoneal injury, (2) prevent coagulation of serous exudates, (3) remove or dissolve deposited fibrin, (4) inhibit fibroblast proliferation of adherent structures, and (5) provide a barrier to separate the repair site and intra-abdominal structures until reperitonealization has occurred. For example, in small tissue repairs, it may be possible to use existing tissue such as existing peritoneal or omentum tissue to surround the prosthetic mesh and provide a barrier to prevent adhesion formation. However, in many cases, the existing tissue may be inadequate in size to completely protect surrounding tissues and organs from contacting the prosthetic mesh. No product has been identified that consistently minimizes the formation of adhesions while maximizing cellular infiltration or neovascularization.

Absorbable meshes made of polygalactin 910 (Vicryl™ (Ethicon Inc., Somerville, N.J.)) and polyglycolic acid (Dexon™) can provide an intact structural repair, but lose tensile strength as they degrade (Yannas et al., J. Biomed. Mater. Res. 14:107-132 (1980); Kateusz et al., PomeryW Medycynie 24:3-39 (1994)). Once degraded, the fibrous tissue response that results does not have the strength to provide ongoing support of the repair and eventually breaks down. The incidence of recurrent herniation in the repair is nearly 100% (Green et al., Surgery Gynecology & Obstetrics 176:213-216 (1993)). For this reason, absorbable mesh cannot provide permanent reconstruction of load-bearing tissue. Additionally, as described by Baykal et al., and Diamond et al., polyglycolic acid mesh and polygalactin mesh actually increased incidence of abdominal adhesions in studies performed using rabbits and mice. Similarly, carboxymethylcellulose and carboxymethylcellulose/sodium hyaluronate (Seprafilm™) was shown to reduce adhesions in a rat ventral hernia model repaired with PP mesh; however, incorporation of the mesh into the abdominal wall was impaired. Similar inconsistent results have been demonstrated using oxidized regenerated cellulose such as Interceed TC7® and Surgicel®. An additional drawback with absorbable wound repair materials has been the speed of degradation. Most absorbable meshes degrade within 5-7 days, prior to adequate cellular infiltration and neovascularization, thus reducing the overall quality of the newly formed tissue.

The ability of a collagen-glucosaminoglycan (CG) matrix to induce the formation of tissue has been well studied in the dermis and nerve (Yannas et al., J. Biomed. Mater. Res. 14:65-81 (1980); Yannas et al., J. Biomed. Mater. Res. 14:107-132 (1980); Dagalakis et al., J. Biomed. Mater. Res. 14:511-528 (1980); Murphy et al., Lab. Invest. 63:305 (1990)). The use of CG matrix as part of a multilayer composition useful as synthetic skin is described in U.S. Pat. No. 4,060,081 to Yannas et al. The Yannas patent refers to a multilayer membrane consisting of a CG matrix layer that is insoluble by body fluids and nonbiodegradable in the presence of body enzymes (col. 3, lines 40-45), in conjunction with a separate, non-integrated moisture transmission control layer necessary to control moisture flux with the external environment. Yannas further refers to the use of an optional third material to provide mechanical reinforcement of the epidermis. The mechanical reinforcement material of Yannas is a separate cotton or other textile mesh that is placed over the CG matrix, covered with the moisture transmission control layer by knife coating and then cured to create the final composition, resulting in a stiffer composite. (col. 13, line 61-col. 14, line 4). The cotton layer of Yannas is used to reinforce the CG matrix to allow easier handling during use. One of skill in the art will recognize that cotton is not a suitable support material for bridging gaps in wounds or tissues. The elasticity and low tensile strength of cotton results in increased scarring and stretching of the wound repair. Further, the multifilament structure of cotton leads to increased inflammation, infection and formation of undesirable fibrotic tissue at the wound site. The compositions of Yannas et al. are specifically referred to as skin replacements for epidermal use. One of skill in the art will readily appreciate that the use of such a layered membrane for repair of non-cutaneous wounds or tissue defects, for example intra-abdominal or peritoneal repairs, can result in many of the complications described herein relating to the use of other prior art materials, including wound infection, seroma and hematoma formation, adhesion and fistula formation, and wound separation.

Nonabsorbable structural meshes composed of polypropylene (PP) (e. g., Marlex™ (C. R. Bard Inc.), Prolene™ (Ethicon Inc., Somerville, N.J.)), Dacron™ (e.g., Mersilene™ (Ethicon Inc., Somerville, N.J.)) and expanded polytetrafluoroethylene (e.g., Gore-tex™ (W. L. Gore and Associates)) have generally been used for increasing structural stability in tissue repair. Although placement of structural meshes directly in contact with abdominal viscera is avoided when possible, this may not be possible in many reconstructions. When applied in the form of a mesh, mechanical properties such as tensile strength, modulus of elasticity, and flexural rigidity can be controlled using a variety of polymers.

PP mesh is the most commonly used prosthetic mesh for tissue defects, and it is ultimately the standard to which materials are compared due to its favorable mechanical properties and biocompatibility. This macroporous mesh is inert, strong, and rapidly traversed by fibrous tissue. Scar tissue that forms around and through the mesh strengthens the repair zone. This tissue infiltration, however, is not well organized and the resulting scar tissue can contract and distort the mesh. Moreover, the outer ends of the mesh contain rigid monofilaments that are sharp and abrasive; these sharp edges have been reported to injure underlying viscera and erode through overlying skin and soft tissue, leading to visceral perforation, fistulization, and infection. PP mesh also causes dense adhesions when it is placed adjacent to the abdominal viscera (Deguzman et al., Endoscopy 27:257-461 (1995)). Complications with the use of PP mesh include wound infection, scarring (Elliot et al., Am. J. Surg. 137:342-344 (1979)), seromas (Gilbert, South Med. J. 80:191-195 (1987)), sinus formation (Molloy et al., Br. J. Surg. 78:242-244 (1991); Boyd, Surg. Gynecol. Obstet. 144:251-252 (1977)), mesh extrusion (Voyles et al., Ann. Surg. 194:219-223 (1981); Lamb, Surg. 93:643-648 (1983)) and fistula formation (Talbert et al., J. Pediatr. Surg. 12:63-76 (1977); Deguzman et al., Endoscopy 27:257-461 (1995)). These complications may lead to more serious problems including bowel obstruction, perforation, and reherniation requiring additional surgical repair.

Work described herein has reaffirmed the high incidence of adhesion formation reported using polypropylene mesh directly exposed to peritoneal contents for ventral hernia repair (Alponat et al., Am. Surg. 63(9):818-819 (1997); Cristoforoni et al., Am. Surg. 62(11):935-938 (1996)). PP graft repairs in this study formed dense adhesions to both the omentum and bowel involving over 70% of the mesh surface involved.

Dacron™ mesh is more flexible than PP and rapidly conforms to anatomical defects. This mesh has not gained widespread use in the United States for several reasons. Dacron™ has been reported to elicit an inadequate fibrous response; several investigators have indicated that the fibrous tissue which grows into Dacron™ mesh becomes only loosely associated with the fibers of the mesh (Johnson-Nurse and Jenkins, Biomaterials 10(6):425-428 (1989)). Dacron™ also causes bowel adhesions and can cause visceral perforation and fistula formation. In addition, Dacron™ has a multifilament construction and has been associated with increased infection rates, as multifilament fibers provide an environment for bacteria to colonize which is relatively inaccessible to macrophages.

Expanded polytetrafluoroethylene is the least reactive of prosthetic materials and produces the least inflammatory response. The microporous structure is smooth, and, unlike PP and Dacron™, does not adhere well to abdominal viscera. This mesh does not optimally integrate into host tissue, however, and investigators have attributed a higher rate of recurrent hernias to this fact. The strength of repairs using expanded polytetrafluoroethylene are ultimately dependent on the strength of suture fixation between the edge of the tissue defect and the prosthetic component. (Amid, et al., J Biomed Mater Res 28: 373-375, (1994), Naim, et al., J Laparoendosc Surg 3: 187-190, (1993))

Many of the problems associated with permanent mesh for tissue reconstruction, such as lack of adequate fixation, adhesion, seroma/hematoma, fistula and scarring are related to the direct interaction of the mesh with the adjacent tissue. The presence of a foreign body in the wound combined with poorly vascularized scar tissue surrounding the mesh also makes it susceptible to infection which can be difficult to eradicate without removal of the mesh.

Thus, there remains a need for a composition with the beneficial effects of reducing adhesions to adjacent structures, increased cellular infiltration and neovascularization, but that does not compromise the strength of the wound in order to aid in wound or tissue closure, and does not increase rates of infection. By combining the support material with a biodegradable barrier material as described herein, a well vascularized mesenchymal tissue layer is rapidly formed which completely surrounds the support material. Many of the complications encountered using materials known in the prior art may be reduced by the formation of a vascularized tissue layer between the support material and the subcutaneous tissue. As newly formed tissue surrounds the support material, it protects the adjacent tissue from perforations, erosion of the support material through the skin and soft tissue, scar and adhesion formation, and trauma leading to bleeding or fluid accumulation.

### SUMMARY OF THE INVENTION

Structural materials used to reconstruct abdominal wall defects restore abdominal wall integrity but may cause adhesions to the underlying abdominal viscera as well as additional problems associated with incomplete or irregular cellular infiltration and neovascularization of new tissue. The present invention demonstrates that integrating nondegradable structural support materials with biodegradable barrier materials reduces adhesions and increases well organized, cellular infiltration and neovascularization, resulting in thicker, healthier tissue development at the repair site. Thus, the composite materials described herein provide wound or tissue closing and healing properties superior to those in the prior art.

The present invention pertains to compositions comprising at least one support material integrated with at least one biodegradable barrier material. Alternatively the composition can comprise two or more different biodegradable barrier materials, one of which can function as a support material.

The support material provides a structural bridge or reinforcement for the wound or defect being repaired. The support material can be an absorbable or nonabsorbable material. For example, the support material can be polypropylene mesh such as Prolene™ (Ethicon Inc., Somerville, N.J.) and Marlex™ (C. R. Bard Inc.); polyester such as Dacron™ and Mersilene™ (Ethicon Inc., Somerville, N.J.), silicone, polyethylene, polyamide, titanium, stainless steel, polymethylmethacrylate, silk, cotton, polyglactic acid such as Vicryl™ mesh (Ethicon Inc., Somerville, N.J.), polyglycolic acid such as Dexon™ mesh, poliglecaprone, collagen, gelatin, polydioxone and expanded polytetrafluoroethylene such as DualMesh™, Mycromesh™ or other expanded PTFE (W. L. Gore and Associates). In certain instances, the barrier materials described below may function as support materials. One of skill in the art can identify barrier materials with the necessary characteristics to function as a support material.

Biodegradable barrier material of the present invention serves as a temporary tissue substitute and template for new tissue formation. The biodegradable barrier material can be, for example, collagen glycosaminoglycan matrix (e.g., a crosslinked collagen glycosaminoglycan matrix), Gelfoam™ (Pharmacia and Upjohn, Inc., Kalamazoo, Mich.), Surgicel™ (Johnson & Johnson), carboxymethylcellulose or carboxymethylcellulose/sodium hyaluronate such as Seprafilm™, oxidized regenerated cellulose such as Interceed TC7® and Surgicel®, acellular cadaveric dermal matrix such as Alloderm® or a particulate form of acellular cadaveric dermal matrix such as Cymetra™. In other embodiments, the support material of the disclosed composition is made of combinations of biological and non-biological materials. Examples include dermis, fascia, tendon, or any other material described herein or recognized by a skilled artisan to be a useful material for support and reinforcement of the tissue repair, in combination with polymeric or other materials as listed above or as would be recognized by a person skilled in the art to be useful

When combined with the support material, the barrier material aids the formation of mesenchymal tissue adjacent to and incorporated in the support material. This orderly, well vascularized tissue grows around and through the support material, providing strength, vascularity, and a healthy barrier layer of tissue to separate the support material from surrounding tissue and organs while fixing the support material in place. This healthy tissue, such as new tissue including new mesenchymal tissue, is distinct from the thin, scar tissue normally associated with wound and tissue repair using materials and methods of the prior art.

Suitable barrier material comprises materials including but not limited to, cellular materials, biologically-derived or synthetically-produced acellular materials or cellular components, or combinations of these. Examples of such materials that can be used include, without limitation, dermal, epidermal, epithelial, muscosal or submucosal tissue or cells, or cellular or non-cellular components of the dermis, epidermis, epithelium, muscosa, or submucosa, including the extracellular matrix, basement membranes, or their analogs, or combinations of any of these. The dermis, epidermis, epithelial, mucosa or submucosa can be decellularized, thus decreasing viral transfer from the graft to the host. Dermal cells, epidermal cells, epithelial cells, mucosal cells or submucosal cells, intact extracellular matrices, intact basement membranes and other acellular structures including analogs, contain a scaffold for cellular infiltration and promote wound healing and tissue repair. Additional barrier materials include pleura, fascia, tendon, dura, peritoneal cells, pericardium, mesothelium, blood vessels, synovial surfaces, joint tissues, fat, and amnionic membrane. Processed or synthetic materials that may be used as a barrier material include decellularized tissue that may or may not include the basement membrane, such as decellularized cadaveric dermis, such as Alloderm® or Cymetra™, soft tissue grafts, such as Surgisis™; bioresorbable hyaluronic-based material such as Seprafilm™, Sepramesh™ and Sepracoat™; carboxymethylcellulose; oxidized regenerated cellulose; gelatin foam such as Gelfoam® or Gelfilm®. Example barrier materials include collagen, particularly collagen-glucosaminoglycan matrices (CG); and decellularized cadaveric dermis such as Alloderm™ or Cymetra™ (LifeCell Corp., Branchburg, N.J.).

The composition can also comprise a temporary optional moisture barrier to prevent evaporation and provide protection from the environment until sufficient epithelial coverage is obtained. The present invention also relates to synthetic tissue comprising a composition according to the invention. Also disclosed are methods for using the composite materials of the invention.

### DESCRIPTION OF THE DRAWINGS

FIG. 1: This figure is a schematic representation of a composition 1, including a barrier material of the composition 2, and a support material of the composition 3.

FIG. 2: This figure is a cross-sectional view of the composition 1 of FIG. 1 along axis A, including a barrier material of the composition 2, and a support material of the composition 3.

FIG. 3: This figure is a schematic representation of one method of making a composition according to the invention. The composition formed by this method is the collagen-glucosaminoglycan/polypropylene mesh composite used in Example 1.

### DETAILED DESCRIPTION OF THE INVENTION

Mammals suffer tissue loss from a variety of mechanisms including trauma, tumor removal, vascular disease, genetic defects, cosmetic surgery and infections. Replacement of lost tissue or organs is often essential for either survival or function of the mammal.

Many mammalian tissues can be thought of as bi-layer constructs. The surface layer contacts the environment or one or more body fluids, and the stromal layer provides mechanical support and a vascular supply to the surface layer(s). These bilayer tissue types include skin, trachea, bronchi, vermillion, oral lining, nasal lining, stomach, intestines, biliary ducts, ureters, bladder and blood vessels. Replacement of these tissues or structures is most effective when both the stromal and surface layers are reconstituted. In the examples below, abdominal hernia repair will be described in detail as an example of a tissue repair. This invention is not construed to be limited to abdominal or intestinal tissue as the appropriate tissue; the invention is intended to encompass any and all of the tissue constructions known in the art, including but not limited to bi-layer tissues.

A mammal who has suffered extensive tissue loss or injury is immediately threatened by infection and by excessive loss of fluids. To address both of these issues, a large wound or tissue defect must be closed promptly by some type of membrane. The most direct method of accomplishing this purpose is to remove the injured tissue and graft a composition to the wound or tissue defect, restoring the function of the injured tissue. By integrating a biodegradable support material into a barrier material adhesion strength, overall adhesion surface area involvement, and degree of bowel involvement are significantly reduced.

Disclosed herein are novel and non-obvious compositions for wound and tissue repair comprising a support material and a barrier material. Compositions of the invention comprising at least one support material integrated with at least one biodegradable barrier material have been developed and tested in a guinea pig model. A method of repairing or regenerating tissue has been developed which optimizes functional repair, isolates or separates the support material from the underlying tissue or organs to minimize adhesion to surrounding tissue, enhances formation of a pronounced fibrovascular infiltration into the composition, and provides a vascularized tissue bed which rapidly and completely surrounds the structural material and readily supports grafted tissue, such as split thickness skin grafts.

Also disclosed are methods for employing the disclosed composition to repair wounds and tissue defects. The barrier material of the disclosed compositions is a material that can be substantially organic or biodegradable, that minimizes formation of adhesions between the internal structures being protected through closure of the wound, and the support material. Once installed, the barrier material is infiltrated with, and in some cases degraded and replaced with, vascularized host tissue, while dense fibrovascular ingrowth incorporates the support material to the edges of the wound or tissue. This provides a solid, reliable repair with limited complications of adhesions and minimization of fluid or air leakage. Thus, the compositions and methods presently disclosed provide structural support for wound or tissue closure and allow dense fibrovascular ingrowth and scarring localized to the synthetic supporting material, minimizing adhesions to organs or structures of the host, without significantly sacrificing the strength or reliability of the wound or tissue repair.

I. The Support Material

The support material of the present invention can be comprised of any materials possessing the strength and structural integrity to promote the integrity of the wound or tissue closure. The composition and structure of the material must be such that it does not provoke a substantial immune response from the mammal in whom it is implanted. The material is preferably permanent and non-biodegradable, particularly for load-bearing tissue, as absorbable materials lose tensile strength as they degrade and the resultant fibrous tissue does not have the strength to provide ongoing support of the repair. For non-load-bearing tissue, the material can be biodegradable but should preferably persist for a period of time sufficient for the formation of new tissue sufficient to support surrounding tissue associated with wound location and tissue function. Characteristics such as pore size, strength, permeability and flexibility can be used to select an optimal material for specific tissue repair or reconstruction. Such optimization is routine and is dependent upon the desired properties of the material and the tissue to be repaired. Desirable characteristics are easily recognized by one of skill in the art and can be determined, for example, with reference to Scales, Proc. Roy. Soc. Med. 26:647 (1953).

One of skill in the art will readily recognize a number of suitable materials to serve as the support material. For example but without limitation, the support material can be made of the host's tissue (in other words, tissue obtained from the subject that has the wound or tissue defect being repaired) or other tissue from an allogenic, homogenic, autologous, xenogenic or synthetic source. In other embodiments, the support material comprises a processed material. Examples of such polymeric or other materials commonly used as support materials include, but are not limited to, polypropylene mesh such as Prolene™ (Ethicon Inc., Somerville, N.J.) and Marlex™ (C. R. Bard Inc.); polyester such as Dacron™ and Mersilene™ (Ethicon Inc., Somerville, N.J.), silicone, polyethylene, polyamide, titanium, stainless steel, polymethylmethacrylate, silk, cotton, polyglactic acid such as Vicryl™ mesh (Ethicon Inc., Somerville, N.J.), polyglycolic acid such as Dexon™ mesh, poliglecaprone, collagen, polydioxone and expanded polytetrafluoroethylene such as DualMesh™, Mycromesh™ or other expanded PTFE (W. L. Gore and Associates).

In other embodiments, the support material of the disclosed composition is made of combinations of biological and non-biological materials. Examples include dermis, fascia, tendon, intestinal submucosal tissue, decellularized cadaveric dermis or any other material described herein or recognized by a skilled artisan to be a useful material, in combination with polymeric or other materials as listed above or as would be recognized by a person skilled in the art to be useful. In certain instances, the barrier materials described below may function as support materials. One of skill in the art can identify barrier materials with the necessary characteristics to function as a support material.

II. The Barrier Material

The biodegradable barrier material can be a highly porous, fibrous lattice. The lattice serves as a temporary tissue substitute and template for new tissue formation and, when combined with support materials, it directs the formation of mesenchymal tissue adjacent to and incorporated in the support material. The adjacent tissue, which is formed from cellular infiltration, neovascularization and/or collagen deposition, is incorporated into the support material by surrounding individual fibers. This orderly, well vascularized tissue grows around and through the support material, providing strength, vascularity and a barrier layer of tissue (unlike scar tissue) to separate the support material from surrounding tissue and organs. The tissue formation also fixes the support material securely to the surrounding tissue.

The composition and structure of the barrier material should be such that it does not provoke a substantial immune response from the graft recipient. The barrier material should be sufficiently porous to permit blood vessels and cells such as inflammatory cells, mesenchymal cells, fibroblasts and other cells from healthy tissue surrounding the wound to migrate into the barrier material. As discussed herein, this migration is referred to as “infiltration” and is responsible for the generation of the new tissue. Appropriate barrier materials can also be selected on the basis of properties such as degradation rate, hemostatic ability, degree of neovascularization, cellular infiltration and scar formation attributed to a particular barrier material. This optimization is routine in the art.

To facilitate the formation of new, non-scar tissue, the barrier material should be biodegradable. This biodegradation must not proceed so rapidly that the barrier material disappears before sufficient healing occurs, i.e. before sufficient infiltration and neovascularization occurs. Barrier materials that degrade too slowly often result in excessive scarring and increased adhesion formation. One skilled in the art can determine the appropriate degradation according to the wound or tissue damage being repaired. Determination of optimal biodegradation periods according to individual circumstances is routine in the art.

Barrier materials of the present invention may be allogenic, homogenic, autologous, xenographic or synthetic in origin. Alternatively, the barrier materials can be made from a combination of these or other sources known to the skilled artisan, or can be synthesized. Allogenic sources include living or deceased humans, so the materials can be cadaveric or living.

Barrier materials comprise cellular materials such as dermal, epidermal, or epithelial cells or tissue such as peritoneal tissue; mucosal or submucosal cells or tissue; acellular materials, such as an intact basement membrane or an acellular mucosal, submucosal, epithelial, epidermal or dermal layer; or any combinations or equivalents thereof.

Dermal tissue, dermal layer cells, epidermal tissue, epidermal layer cells, epithelial tissue including peritoneal tissue, or epithelial cells including peritoneal cells can be used to form all or part of the barrier material of the present invention, alone or in combination with any other suitable barrier material as will be recognized by one of skill in the art. Dermal, epidermal or epithelial cells that are useful include glands, vascular cells or networks, fibroblasts, and keratinocytes. Additional dermal, epidermal, or epithelial cells that are useful will be apparent to the skilled artisan. The dermal, epidermal, or epithelial tissues or cells that can be used can be derived from a wide variety of sources such as human or animal sources.

Alternatively or additionally, mucosal or submucosal tissue or cells can be used as barrier material, alone or in combination with any other suitable barrier material as will be recognized by one of skill in the art. Mucosal or submucosal cells that can be used to form all or part of the barrier material include any connective tissue cells, such as those, which when obtained from naturally occurring source, are found in the intestinal tract, such as the esophagus, stomach, large intestine or small intestine; the urogenital tract, such as the bladder; the reproductive tract, such as the uterus; or from other organs such as the pericardium. Specific mucosal or submucosal cells that are useful include glands, fibroblasts, smooth muscle cells, gastric cells, uro-epithelial cells, respiratory epithelial cells, or oral or vascular endothelial cells. Additional mucosal or submucosal cells that are useful will be apparent to the skilled artisan. The mucosal or submucosal tissues or cells that can be used can be derived from a wide variety of sources, or combinations of sources, such as the intestine, bladder, stomach, blood vessels, and the like, and may be obtained from humans or other animals.

Alternatively or additionally, a variety of other tissues or cells can be used as barrier material, alone or in combination with any of the other suitable barrier materials as will be recognized by one of skill in the art. Such tissues or cells that can be used include pleura, fascia, tendon, dura, pericardium, mesothelium, blood vessels, synovial surfaces, joint tissues, fat, and amnionic membrane.

Alternatively or additionally, acellular structures can be used as all or part of the barrier material of the present invention, alone or in combination with any of the other barrier materials. Examples of acellular structures that are useful as the barrier material include, but are not limited to, the extracellular matrix, or ground substance, or any other matrix, including those matrices composed of polysaccharides and proteins. In general, but not necessarily, the proteins included in useful matrices will be fibrous or adhesive or elastic, or some combination of these, so that the barrier material naturally forms a membrane or connective structure, or can be engineered or formulated to form a membrane or connective structure. Specific acellular structures that can be used to form all or part of the barrier material include, but are not limited to the following, either alone, or in combination with other acellular structures, dermal tissues or cells, or submucosal tissue or cells: basement membrane, fibrin, laminin, hyaluronic acid, bamacan, heparin sulfate proteoglycan, perlecan, agrin, collagen, or intactin.

Other examples of materials that can be used as all or a part of the barrier material, alone or in combination with each other or the other materials described as being useful components of the barrier material include: decellularized tissue that may or may not include the basement membrane, such as decellularized cadaveric dermis, such as Alloderm®; soft tissue grafts, such as Surgisis™; bioresorbable hyaluronic-based material such as Seprafilm™, Sepramesh™ and Sepracoat™; carboxymethylcellulose; oxidized regenerated cellulose such as Interceed TC7® and Surgicel®; gelatin foam such as Gelfoam® or Gelfilm®; peritoneal cells; fascia; pleura; dura; pericardium; tendon; or blood vessels.

A preferred barrier material is acellular dermal matrix such as decellularized cadaveric dermis marketed under the tradename Alloderm™ by LifeCell Corp., Branchburg, N.J. Cadaveric donor tissue is collected and epidermal material is removed while preserving the underlying dermis. This dermal tissue is then treated to denature and remove dermal cells while retaining the structural integrity of the dermal scaffold such as channels for vascularization, collagens, proteoglycans and elastin structures necessary for proper cellular infiltration and neovascularization. Additionally, basement membrane components, including laminin and collagen types IV and VII remain intact and attached to the surface, enhancing the infiltration, proliferation and attachment of epithelial cells during healing. Decellularized cadaveric dermis may be in sheet form such as Alloderm™ or it may be in particulate form such as Cymetra™.

A second preferred barrier material is collagen-glucosaminoglycan matrix (“CG matrix”). CG matrix is a highly porous lattice made of collagen and glycosaminoglycan. The CG matrix serves as a supporting or scaffolding structure into which blood vessels and mesenchymal cells infiltrate, creating new mesenchymal tissue which replaces the CG matrix as it biodegrades. Cells from undamaged tissue surrounding the edges of the wound migrate into the CG matrix to create a new, vascularized tissue bed.

Function of the CG matrix is likely to be influenced by other physiochemical properties such as the type of glycosaminoglycan (GAG) used, the concentration of GAG, the pore structure, the collagen density, and the ability of collagen to activate platelets. These properties can be optimized using routine methods known to the skilled artisan. Various forms of GAG which may be suitable for use in this material include chondroitin 6-sulfate, chondroitin 4-sulfate, heparin, heparin sulfate, keratin sulfate, dermatan sulfate, chitin and chitosan.

It is possible to control several parameters of the CG matrix (primarily crosslinking density, porosity and GAG content) to control the rate of biodegradation of the lattice. Increasing collagen crosslink density by gluteraldehyde treatment, making alterations in the composition of the CG matrix, or using other glycosaminoglycans such as heparin or hyaluronic acid could affect the biodegradation, enhance antiadhesive properties or affect other desired properties of the composition. The skilled artisan will appreciate other specific conditions indicating the use of CG matrices with variations of the above-mentioned parameters which are suitable for use in the present invention. In addition, certain applications of tissue regeneration may require matrices which degrade more slowly or more quickly. The skilled artisan will be able to recognize applications where it is desirable to vary the properties of the CG matrix, and will be able to vary the parameters accordingly and the present invention is intended to encompass such variations.

More than one layer of barrier material can be used to make up the composite. This allows for varying thickness of the barrier depending on the type of wound or tissue closure desired. For example, two, three, four, five or even more layers of barrier material can be used. Additionally, when multiple layers of barrier material are used, the layers can be made from the same material or combinations of materials, or the layers can be made from different suitable barrier materials. For example, each layer can be the same barrier material or combination of materials as the other layers, two or more can be the same, or they can all be different, according to the needs of the individual, or the wound or tissue being repaired.

III. Composite Structure

The compositions of the invention can comprise two or more layers in accordance with the teachings herein. For example, at a minimum the composition comprises one layer of biodegradable barrier material and one layer of support material. The composition can also comprise three layers, wherein the support material is disposed between and completely integrated with two layers of biodegradable barrier material. One benefit to the completely integrated composition is that the outer barrier material provides the ability to separate the support material from surrounding tissue, and the inner barrier material provides a vascularized bed which will support grafted tissue, help fill dead space or contour irregularities. Grafting of tissue can be performed using any of several methods known in the art. Additional layers of biodegradable barrier material and support material can also be incorporated as desired to improve the properties of the compositions. The biodegradable barrier materials used can be the same or different. In a preferred embodiment, one or both of the biodegradable barrier material layers is larger than the support material to allow the barrier material to surround the support material and prevent it from contacting the surrounding tissue. Compositions comprising support materials and biodegradable barrier materials can be constructed by methods described herein or by other methods known in the art.

IV. Composite Processing

In another embodiment, the present invention provides a composition that has been specifically constructed to have various characteristics useful in different applications, according to the needs of the artisan employing the compositions or methods disclosed herein, or the wound or tissue being repaired. For example but without limitation, the composition can be designed so that either or both materials have one or more of the following properties: anti-adhesive; antibiotic; anti-viral; anti-fungal; anti-thrombotic; pro-thrombotic (hemostatic); immunosuppressive; anti-inflammatory; wound-healing-promoting or suppressing; angiogenic or anti-angiogenic. One skilled in the art will readily recognize the many substances available to confer these and other useful properties to the disclosed compositions. For example, but without limitation, examples of anti-adhesive substances that could be added to the materials of the disclosed compositions include, but are not limited to, heparin or anti-thrombolytics, which include streptokinase, urokinase, tissue plasminogen activator, or other defibrinogenating enzymes such as ancrod (marketed under the tradename Viprinex™ by Knoll Pharmaceuticals). Anti-inflammatory agents that could be used include steroids, non-steroidal anti-inflammatory agents, and chemotherapeutic agents. Enhanced wound-healing properties can be achieved through the use of any of the known growth factors such as, without limitation, vascular endothelial growth factors, platelet-derived growth factors, epidermal growth factors, insulin-like growth factors, transforming growth-factor beta, or fibroblast growth factor. Suppressed wound-healing can be achieved through use of any of the known growth factor suppressors.

Bathing, injecting, transfecting, bonding, coating, adding genetically modified cells and/or genetic material itself, and laminating are a few ways that the anti-adhesive or other substances conferring desirable properties can be added to the materials of the disclosed compositions. Peritoneal or epithelial cells or any other cells or cell components that may reduce adhesions, enhance the strength of the repair, or provide other desirable charcteristics may also be added to the composite. Cells or tissue could be cultured, seeded, grafted, injected, or layered into the materials of the composition.

When repairing epidermal wounds or tissue damage, compositions of the invention can also comprise an optional moisture barrier, such as an impermeable silicone surface layer, which can provide a temporary border or cutaneous reconstruction to prevent evaporation and provide protection from the environment while the epithelial layer is forming and becoming confluent. The optional moisture barrier is any material which can serve as an outer surface to the composition and should be capable of being absorbed after a suitable period of time or manually removed at will from the composition. Materials suitable for use as a moisture barrier must also have the property of being semipermeable to the passage through the wound of fluids from inside the body and impermeable to microorganisms such as bacteria and viruses from outside the body. The moisture barrier layer may not be necessary for internal uses or other applications such as, for example, those in which the tissue or organ is not exposed to the external environment, and thus it is optional in such applications. Silicone elastomers are suitable for use in the moisture barrier of the present invention.

V. Attachment of Barrier Material to Supporting Material

The barrier material may be attached to, integrated around, or placed onto the support material by a wide variety of means. Examples of such means are simply placing the barrier material over the supporting material or physically attaching the barrier material to the supporting material by means such as but not limited to, bonding, including by using adhesives such as cyanoacrylate or other types of adhesives or glue, fibrin glue, fibrin, thrombin, plasma, or cellular derived hemostatic/adhesive agents; mechanic agents such as suturing or stapling; or laminating. In certain embodiments, the support material may be encased by barrier material such that the support material is substantially surrounded by and integrated with the barrier material. Other means of attaching the supporting material to the barrier material or layers will be readily apparent to those skilled in the art.

VI. Wounds or Tissue Defects to be Repaired

Compositions according to the present invention can be used to repair any type or size of wound or tissue defect. Examples include but are not limited to repairing pelvic defects, joint defects, abdominal defects, chest wall defects, cranial defects, hernias, congenital abnormalities, skin lesions, bums, surgical incisions or traumatic wounds.

The present invention has application to massively burned patients as well as to patients undergoing reconstructive surgery, tissue trauma, surgical resection, infection, chronic skin diseases and chronic wounds. The present invention will also be useful in the replacement of other specialized epithelial tissues in a variety of organ systems, including but not limited to, bone, cartilage, oral mucosa, uroepithelial, gastrointestinal, respiratory and vascular. Tissue loss from malignancy, congenital or acquired disease and surgical removal can be replaced with tissue composed of the same specialized native cells. Specialized epithelial tissue such as bladder, ureter, oral mucosa, esophagus, trachea, blood vessel and intestine often requires replacement or reconstruction after surgical excision.

Compositions described herein can be used by the oncologic, trauma or reconstructive surgeon to replace tissue defects with a tissue composed of organ-specific cells identical to the native tissue, without the need to violate uninjured organs for donor tissue. Such tissue can be replaced after surgical resection for malignancy, disease or trauma. This method allows for replacement of various commonly lost tissues such as oropharyngeal, nasal and bronchial mucosa, lip vermillion, blood vessels, trachea, esophagus, stomach, small and large bowel, biliary ducts, ureter, bladder, urethra, periosteum, synovium, areolar tissue, chest wall, abdominal wall and vaginal mucosa. Structural defects such as ventral, inguinal and diaphragmatic hernias, replacement or augmentation of tendons, ligaments and bone and abdominal and thoracic wall reconstruction can also be repaired as described herein. The composition is flexible enough to be molded into the appropriate shape or form and then secured to adjacent or contiguous uninjured tissue while tissue regeneration progresses. One of skill in the art will readily recognize alternative and various types of wounds or tissue defects for which the present compositions and methods will be useful.

VII. Methods of Using the Compositions

Once the composition has been prepared the wound or tissue is readied for application of the composite. Areas of tissue that have been destroyed or damaged are surgically removed to prevent them from interfering with the healing process. The entire area of dead and damaged tissue is excised, so that intact epithelial cells are present at the perimeter of the wound or tissue. The composition, with the optional moisture barrier, if present, away from the wound or tissue, is draped across the wound to avoid the entrapment of air pockets between the wound or tissue and the composition. The composite is sutured or stapled to the wound or tissue using conventional techniques and the wound or tissue is then covered or closed, as appropriate.

After application of the composition to the wound or tissue, blood vessels, inflammatory cells, fibroblasts and other epithelial and mesenchymal cells from underlying healthy tissue begin, as described herein above, the process of infiltration of the grafted composite. Once the infiltration has progressed sufficiently to the point where the replacement tissue can function to protect the body against infection or infiltration from micro-organisms and moderate fluid passage, the optional moisture barrier (if present) is manually removed or is absorbed from the composite.

For example, abnormal tissue can be intentionally (e.g., surgically) removed from an individual and new tissue can be elicited in its place using this method. Alternatively, the method of the present invention can be used to produce new tissue in place of tissue which has been lost due to accident or disease.

In one embodiment, the present invention provides a method for repairing wounds or tissue defects by employing the disclosed composition to promote strength of a wound or tissue closure. In an alternative embodiment, the present invention provides methods for preventing adhesion of a composite to undesired organs or other structures (or both) of the host. In another embodiment, the present invention provides a method for promoting the formation of a tissue layer at the site of a wound or tissue repair, by using the compositions disclosed herein. In this embodiment, the tissue layer will generally form between the barrier material, which can be dissolved over time, and the support material. The composition can be stapled, sutured, glued, or otherwise placed in the patient to repair the wound or tissue defect. Other alternative forms of placement of the composite for wound or tissue repair are also available and will be readily appreciated by one of skill in the art.

The following Examples are offered for the purpose of illustrating the present invention and are not to be construed to limit the scope of this invention. The contents of all references, patents and published patent applications cited throughout this application are hereby incorporated herein by reference.

### EXAMPLES

### I. Example 1

Polypropylene Mesh Supporting Material Integrated with and Encased by Collagen-Glucosaminoglycan Barrier Material

A. Materials and Methods

Hartley guinea pigs underwent ventral hernia repair with either PP mesh alone (control), or PP mesh encased within a collagen-glucosaminoglycan (CG) matrix to form a composite according to the present invention. Gross and histologic observations were made at 4 weeks. Strength, surface area involvement by adhesions, and histologic appearance of the repair sites are compared.

1. Graft Preparation

Bovine hide collagen (Sigma Chemical Co., St. Louis, Mo.) 0.5% by weight was dispersed in 0.05M acetic acid and co-precipitated with chondroitin-6-sulfate (Sigma Chemical Co., St. Louis, Mo.). The co-precipitate was concentrated by centrifugation and excess acetic acid was decanted. Concentrated co-precipitate, 3 ml, was poured into 3×5 cm wells on a flat stainless steel freezing pan placed on a cooled (−30° C.) shelf of a freeze-drier. After the first freeze cycle, polypropylene (PP) mesh (Prolene®, Ethicon Inc., Somerville, N.J.) (2×4 cm) was placed over the collagen-glucosaminoglycan (CG) mesh and 3 additional ml of the CG co-precipitate was poured over the mesh. After a second freeze cycle performed at −30° C., the frozen composite was then sublimated at 200 milliTorr to produce a highly porous composite completely surrounding the PP mesh. The collagen fibers of the matrix were cross-linked using a 24 hour dehydrothermal treatment at 105° C. and 30 milliTorr. Additional cross-linking was performed in selected PP/CG composites using a 24 hour treatment with a 0.25% gluteraldehyde (GA) solution in 0.05 M acetic acid. The composite mesh was then exhaustively dialyzed in sterile, de-ionized water and stored in sterile 70% isopropanol until use. Alcohol was removed immediately prior to implantation by sequential washing with phosphate buffered saline. The hydrated CG/PP graft was approximately 3 mm in thickness.

2. Animal Model

Animals were housed in a facility approved by the Association for Assessment and Accreditation of Laboratory Animal Care (ALAC) and cared for under an approved institutional protocol. Forty female Hartley guinea pigs, 500-525 g, were used in the study. The animals were anesthetized using Halothane (2.5 mg %), oxygen (4 L/min), and nitrous oxide (3 L/min). Electric shears were used to remove the hair from the abdominal wall, which was then prepped with betadine solution and draped sterilely. A 3 cm vertical midline incision centered between the xyphoid and pubis was made through the linea alba and peritoneum to expose the peritoneal cavity. A PP or CG/PP mesh was placed within the peritoneal cavity dorsal to the abdominal wall and peritoneum. Twelve animals were used for PP mesh implantation. Additionally, 14 animals were implanted with CG/PP composites which had been GA crosslinked, and 14 animals were implanted with CG/PP composites without GA cross-linking.

The edge of the abdominal wall defect was sutured directly to the edges of the implants with a running 5/0 nylon suture. This repair resulted in an elliptical fascial defect (3×1 cm) bridged by the implant with a 0.5 cm overlap at all edges. A running 5/0 nylon suture was used to close the skin and the incision was dressed with petroleum impregnated gauze and an elastic bandage. Dressings were removed at 7 to 14 days and wounds left open to air.

3. Analysis

At four weeks, the animals were sacrificed and the entire abdominal wall was circumferentially incised to the peritoneal cavity to widely expose the repair site. The exposure was performed gently to avoid disturbing any adhesions to viscera or omentum. Photographs were taken and observations were scored in a blinded fashion. The structures adherent to the mesh were recorded. These included omentum, small intestine, large intestine, stomach and liver. The percent surface area of mesh involving adhesions was estimated by visual inspection. Adhesion strength was scored qualitatively from 0-3, with 0=no adhesions, 1=adhesions easily freed with gentle tension, 2=adhesions able to be freed with blunt dissection and 3=adhesions requiring sharp dissection to separate from graft site. A transverse section of full-thickness abdominal wall including attached viscera was fixed in 10% formalin, embedded in paraffin and sectioned for staining with hematoxylin and eosin. Results were expressed as mean±standard deviation. Comparisons were made using an unpaired Student's t-test.

4. Results

There were three anesthesia-related deaths (2 in the PP group and 1 in the CG/PP group) and ten animals (1 in the PP group and 9 in the CG/PP group) were excluded after these animals damaged the dressings and skin sutures resulting in skin dehiscence.

In the remaining animals (n=27), adhesions to the materials at 4 weeks were significantly more in the PP mesh (n=9) group than in the CG/PP (n=18) composite group. Some omentum was adhered to both CG/PP and PP implants. Small bowel, however, was adherent to only 3 of the 18 PP/CG repairs but to 8 of 9 PP repairs. The average adhesion score was less in the CG/PP (1.7±0.5) than the PP (3.0±0.0) (p=1.6×10-s). The amount of the material surface area covered by adhesions was also less with the CG/PP composite (20±15%) than with the PP (73±16%) (p=8.2×10-9) (Table 1).

Histological examination of the CG/PP mesh at 28 days showed the polypropylene mesh surrounded with a partially degraded CG material layer. The CG material was infiltrated with cells and was vascularized. This vascularized, mesenchymal tissue layer expanded through the interstices of the mesh fibers, encasing the polypropylene mesh with a continuous tissue layer which extended below the PP mesh an average of 0.34±0.30 mm.

Using PP implants, the polypropylene was directly in contact with the abdominal viscera with dense adhesions. A discontinuous layer of scar tissue formed between the polypropylene mesh and the abdominal viscera which was only 0.05 mm 0.02 mm in thickness. In multiple locations along the mesh, the fibers were directly adjacent and adhered to bowel.

Of the 18 animals in the CG/PP group, 9 animals received CG/PP mesh which underwent additional collagen crosslinking with GA, and 9 received CG/PP composites without GA cross-linking. Both groups, GA treated and untreated, formed significantly fewer, less dense adhesions and a thicker tissue layer in comparison to PP repairs (Table 2). In non-GA crosslinked materials there was only 18% average surface area involvement, adhesion grade of 1.8, and the thickness of the tissue below the mesh was 0.13 mm (Table 3). Within the CG/PP group, comparisons were made between GA crosslinked and non-GA crosslinked mesh repairs (Table 4). No significant difference was observed in adhesion grade (p=0.54) or percent surface area (p=0.35) when these subgroups were compared to each other. The tissue layer that formed under the polypropylene, however, was thicker in GA crosslinked CG/PP composite mesh repairs (0.68±0.14) with than those without GA crosslinking (0.13±0.04), p=0.018. Qualitatively, there were more residual CG matrix fibers observed in GA treated CG/PP mesh repairs at day 28.

TABLE 1
Comparison of CG/PP to PP Mesh

| | PP (n = 29) | CG/PP (n = 18) | p-value |
|---|---|---|---|
| Surface area | 73 ± 16 | 20 ± 15 | 8.2 × 10⁻⁹ |
| involved (%) | | | |
| Adhesion grade | 3.0 ± 0.0 | 1.7 ± 0.5 | 1.6 × 10⁻³ |
| Thickness of tissue | 0.05 ± 0.02 | 0.34 ± 0.30 | 8.0 × 10⁻³ |
| below mesh (mm) | | | |

TABLE 2
Comparison of GA crosslinked CG/PP to PP mesh

| | PP | CG/PP crosslinked | p-value |
|---|---|---|---|
| Surface area | 73 ± 16 | 22 ± 19 | 1.5 × 10⁻⁵ |
| involved (%) | | | |
| Adhesion made | 3.0 ± 0.0 | 1.6 ± 0.5 | 3.9 × 10⁻⁷ |
| Thickness of tissue | 0.05 ± 0.02 | 0.68 ± 0.14 | 8 × 10⁻³ |
| below mesh (mm) | | | |

TABLE 3
Comparison of Non-GA treated CG/PP to PP mesh

| | PP | Non-GA treated | p-value |
|---|---|---|---|
| Surface area | 73 ± 16 | 18 ± 10 | 1.9 × 10⁻⁷ |
| involved (%) | | | |
| Adhesion grade | 3.0 ± 0.0 | 1.8 ± 0.0 | 3.3 × 10⁻⁷ |
| Thickness of tissue | 0.05 ± 0.02 | 0.13 ±0.04 | 8.0 × 10⁻³ |
| below mesh (mm) | | | |

TABLE 4
Comparison within CG/PP mesh group (GA treated vs. untreated)

| | GA Crosslinked | Non-GA Crosslinked | p-value |
|---|---|---|---|
| Surface area | 22 ± 19 | 18 ± 10 | 0.54 |
| involved (%) | | | |
| Adhesion grade | 1.6 ± 0.5 | 1.8 ± 0.0 | 0.35 |
| Thickness of tissue | 0.68 ± 0.14 | 0.13 ± 0.04 | 0.02 |
| below mesh (mm) | | | |

### II. Example 2

Alloderm Barrier Material with PP Mesh Supporting Material Reduced Adhesions in Two Animals

A. Materials and Methods

Hartley guinea pigs underwent ventral hernia repair with one of three meshes: PP mesh alone (control), Alloderm™/PP composite mesh with the dermal basement membrane oriented toward the peritoneal cavity, and Alloderm™/PP composite mesh with the basement membrane oriented away from the peritoneal cavity (i.e., toward the mesh). Gross and histologic observations were made at 4 weeks. Strength, surface area involvement by adhesions, and histologic appearance of the repair sites are compared.

1. Graft Preparation

Prolene® mesh (Ethicon, Somerville, N.J.) implants, 2×4 cm, were used for the PP-only implants and as the PP component of the composite implants. Alloderm™, 7/1000 to 20/1000 inch in thickness, were rehydrated in sterile saline and then cut into 2.5×4.5 cm sections. The Alloderm™ material was sutured over the surface of the PP mesh implants using interrupted 6/0 Vicryl® (Ethicon) sutures. The Alloderm™ material was wrapped around the edges of the PP to completely cover one surface of the mesh as well as its edges. The basement membrane surface of the Alloderm™ was oriented either facing or opposing the mesh. In this example, the basement membrane was opposing the mesh and therefore facing the peritoneal contents.

2. Animal Model

Animal experiments were conducted in an American Association for the Accreditation of Laboratory Animal Care approved facility. The research protocol was reviewed and approved by The University of Texas M. D. Anderson Cancer Center Institutional Animal Care and Use Committee. Hartley guinea pigs (500-600 g) were sedated with buprenorphine (0.05 mg/kg intramuscularly) and then anesthetized with isoflurane (0.5-2%) and oxygen (2 L/min) by mask. All animals received (endofloxacin 5 mg/kg intramuscularly) preoperatively and then once per day for 2 additional days. Electric shears were used to remove the hair from the abdominal wall for each guinea pig. The animal was placed in a supine position, prepared with providone-iodine solution, and sterilely draped. Three-centimeter long midline ventral hernia defects centered between the xiphoid and pubis were created, with incision through the ventral midline skin, subcutaneous fat, linea alba, and peritoneum. Composite mesh implants or control implants were inserted into the peritoneal cavity with the Alloderm™ material facing the peritoneal cavity. The linea alba was sutured to the mesh implants using a running 5/0 Prolene® suture, resulting in an elliptical, 3×1 cm defect in the abdominal wall bridged by the implant alone. The implant was positioned completely intraperitoneally to facilitate adhesions. The skin was closed with stainless steel clips. Animals were monitored until fully recovered from anesthesia and then individually housed. Skin clips were removed 1 week postoperatively.

3. Gross Analysis

At 4 weeks postoperatively, the animals were sacrificed in carbon dioxide chambers. Adhesions were analyzed by circumferentially incising the entire abdominal wall to the peritoneal cavity, to widely expose the repair site for analysis without disrupting the abdominal adhesions. All measurements were performed by experienced observers blinded to the type of implant. For each guinea pig, the surface area of the mesh implant involved with adhesions was assessed. Adhesion strength was graded from 0 to 3 with integrals of 0.5; where 0=no adhesions, 1=adhesions easily freed with gentle tension, 2=adhesions freed with blunt dissection, and 3=adhesions requiring sharp dissection to be freed from the implant site. The intraperitoneal structures/organs involved with adhesions to the repair site were recorded. Evidence of infection, perforation, bowel obstruction, and/or fistulization was noted.

4. Histologic Analysis

A transverse section of full-thickness abdominal wall, including the repair site, adjacent abdominal wall, and attached viscera, was excised from each guinea pig, fixed in 10% formalin, embedded in paraffin, sectioned at 4 μm thick, and stained with hematoxylin-eosin stain. The histologic appearance of the repair site was analyzed using computer-aided planimetry. The degree of dermal degradation, neovascularization, and cellular infiltration was assessed. The cellular composition of the infiltrate within and surrounding the Alloderm™/PP and PP (control) mesh repair sites was determined, as well as the cellular and extracellular composition of the neoperitoneum. The density of cellular infiltration within the Alloderm™/PP graft was quantified and compared between groups. The thickness of the tissue layer beneath the mesh and that of the neoperitoneum as well as that of the neoperitoneum itself was determined and compared.

Sections from each group underwent immunohistochemical staining for basement membrane components and peritoneal cells. Immunostaining for human laminin and type IV collagen were done to determine whether human basement membrane components from the implanted Alloderm® remain. AE1/AE2 anti-cytokeratin immunostaining (staining of peritoneal cells) were done to determine the degree of reperitonealization of the repair site.

5. Statistical Analysis

The number of repair sites with adhesions to the bowel was compared between groups using chi-square analysis. Adhesion surface areas and strengths and the thicknesses of the sub-mesh tissue layer and neoperitoneum were compared using the Mann-Whitney test. A P value below 0.05 is considered statistically significant.

B. Results

Two animals were studied in accordance with the described protocol for model development, and determination of the antiadhesive properties of the Alloderm®/PP composite implant. One ventral hernia defect was repaired with PP mesh (control) and one with an Alloderm®/PP composite implant (with the basement membrane facing the peritoneum). The surface areas of adhesions were 83% and 19% for the PP and Alloderm®/PP repairs, respectively. Moreover, the adhesion strengths were grade 3.0 and 0.5, respectively. There was significant bowel adherence to the PP repair site but none to the Alloderm®/PP site. Histologic analysis demonstrated a dense scar layer beneath the PP repair site, with an extensive amount of small intestine firmly adherent to the mesh. In the Alloderm®/PP repair site the Alloderm® was incompletely degraded, highly vascularized, and densely infiltrated with cells. This vascularized tissue layer had a mean thickness of 444 μm and separated the mesh from the peritoneal cavity. The PP mesh repair sites had PP fibers directly adherent to bowel with an inconsistent scar layer between PP mesh and peritoneal contents with a mean thickness of 52 μm.

This data demonstrate the dramatic antiadhesive properties of this Alloderm®/PP composite implant. Furthermore, the Alloderm® is replaced by highly vascularized host tissue that is incorporated into the PP mesh and separates it from the intraperitoneal structures.

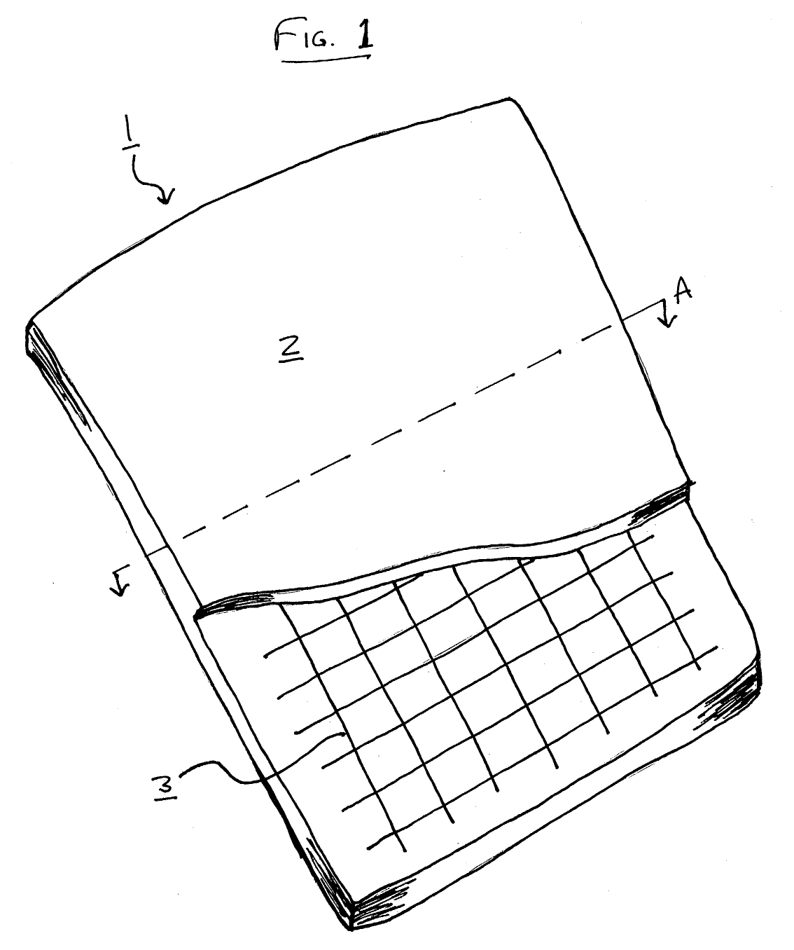

FIG. 1 illustrates the synthetic mesh 1 and a composition 10. The composition 10 comprises a barrier membrane of Alloderm™ 12 and a supporting membrane that, in this embodiment, comprises a synthetic material 14. The barrier membrane 12 covers the edges 16 of the synthetic material 14 since the rough edges 16 of the synthetic mesh are the typical sites for adhesion formation. The synthetic material 14 becomes incorporated with the patient's tissue, and this creates the strength of the wound closure. The composition 10 has the advantage of having a strong closure with decreased adhesions as compared to the prior art 1.

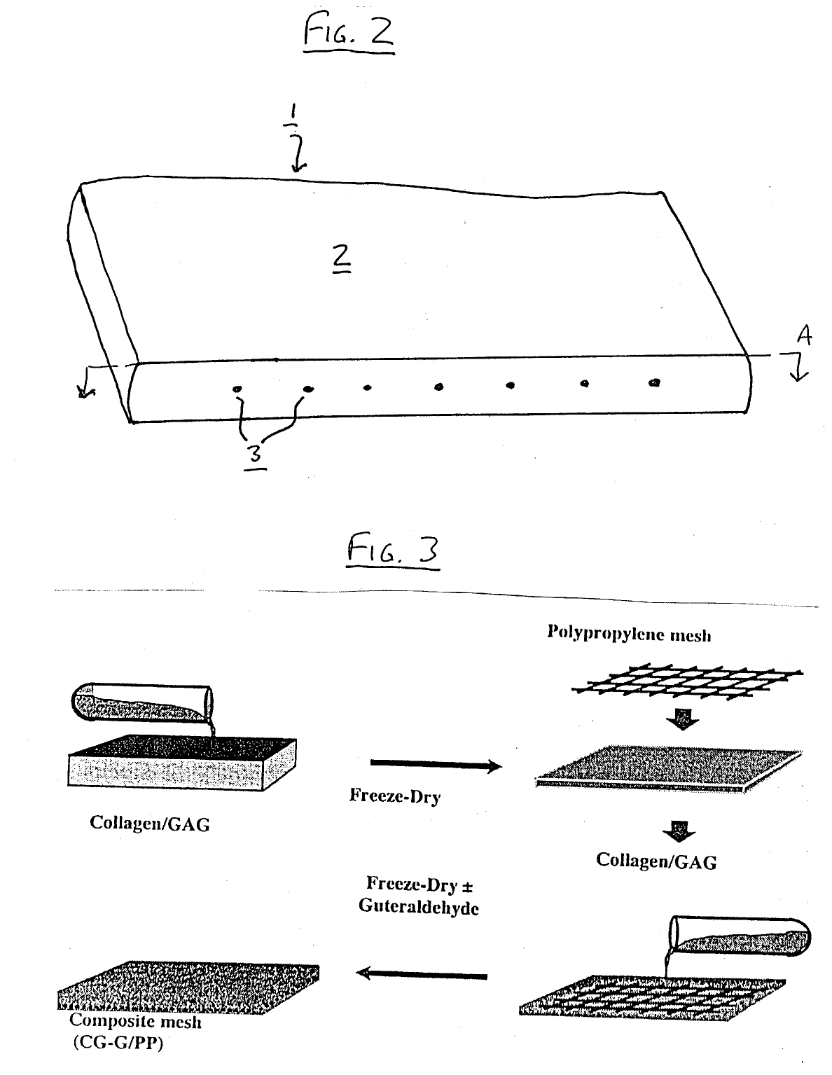

FIG. 3 illustrates an intra-abdominal wound four weeks after composition 34 implantation. As compared to the adhesions 26 in FIG. 2, the adhesions 30 in FIG. 3 are substantially decreased. The barrier membrane 32 is partially degraded and replaced with host tissue and is vascularized.

### III. Example 3

Alloderm Barrier Material with PP Mesh Supporting Material Composition Reduced Adhesions in Larger Studies

A. Materials and Methods

The same materials and methods were employed as with Example 1.

B. Results

Nineteen animals were studied in accordance with the described protocol for model development, and determination of the antiadhesive properties of the Alloderm®/PP composite implant. One ventral hernia defect was repaired on each of six animals with PP mesh (controls), six different animals with an Alloderm®/PP composite with the Alloderm basement membrane facing the mesh (PP/AlloOut), and seven different animals with an Alloderm®/PP composite with the Alloderm basement membrane facing the peritoneum (PP/AlloIn). The average surface area of adhesions for the control group was 79.5±6.1%. The average surface area of adhesions for the two Alloderm®/PP groups were 9.5±12.1% for the PP/AlloOut group and 12.4±8.3% for the PP/AlloIn group. The adhesion strengths for the control group had an average of 2.9±0.20 on the grading scale. The adhesion strengths for the two Alloderm®/PP groups were 0.5±0.45 for the PP/AlloOut group and 1.0±0.41 for the PP/AlloIn group. There was, however, no statistically significant difference in either adhesion surface area or grade between the PP/AlloIn and PP/AlloOut groups. All repair sites in each group involved the greater omentum. The incidence of bowel adherence to the repair site was significantly greater with PP repairs (72%) than the PP/AlloOut (0%) or the PP/AlloIn (0%) repairs.

All implants were rigidly incorporated into the musculofascial edges of the repair sites with dense fibrovascular infiltration around the PP fibers and through the interstices. The vascularized tissue layer that formed beneath the polypropylene mesh at the repair site was significantly thicker in both the PP/AlloIn (634±175 μm) and PP/AlloOut (541±161 μm) than the PP (52±6 μm) group. In addition, the Alloderm®/PP composites were further characterized by highly vascularized host tissue incorporating and replacing the Alloderm® over the 4 weak healing period. At the end of the study, histological examination revealed significant portions of Alloderm® remain incorporated at the wound site, unlike other absorbable meshes that are completely degraded five to seven days after repair. The reduced degradation of the Alloderm® allows for a slower, more regular infiltration and neovascularization of the repair site.

The foregoing descriptions of the invention are intended merely to be illustrative thereof and other embodiments, modifications, and equivalents of the invention are within the scope of the invention recited in the claims appended hereto. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

Claims for Composite Material for Wound Repair:

1. A composition for wound repair comprising a biodegradable barrier material integrated with a support material.

2. The composition of claim 1, wherein the barrier material is comprised of dermal or epithelial tissue.

3. The composition of claim 2, wherein the dermal tissue is selected from the group consisting of epithelium, glands, vascular cells, vascular networks, fibroblasts, and, keratinocytes.

4. The composition of claim 1, wherein the barrier material is comprised of mucosal tissue.

5. The composition of claim 4, wherein the mucosal tissue is selected from the group consisting of esophagus, stomach, large intestine, small intestine, bladder, uterus, pericardium, glands, fibroblasts, smooth muscle cells, gastric cells, uro-epithelial cells, respiratory epithelial cells, oral endothelial cells and vascular endothelial cells.

6. The composition of claim 1, wherein the barrier material is comprised of tissues or cells selected from the group consisting of pleura, fascia, tendon, dura, pericardium, mesothelium, blood vessels, synovial surfaces, joint tissues, fat, and amnionic membrane.

7. The composition of claim 1, wherein the barrier material comprises acellular structures.

8. The composition of claim 7, wherein the acellular structure is selected from the group consisting of extracellular matrix, ground substance, polysaccharide matrix, protein matrix, basement membrane, fibrin, laminin, hyaluronic acid, bamacan, heparin sulfate proteoglycan, perlecan, agrin, collagen, intactin, decellularized tissue including the basement membrane, decellularized tissue excluding the basement membrane, particulate decellularized tissue, a soft-tissue graft, bioresorbable hyaluronic-based material, carboxymethylcellulose, oxidized regenerated cellulose, and gelatin foam.

9. The composition of claim 1, wherein the barrier material comprises a collagen-glucosaminoglycan matrix.

10. The composition of claim 1, wherein the support material is selected from the group consisting of host tissue, allogenic tissue, homogenic tissue, autologous tissue, and xenogenic tissue.

11. The composition of claim 1, wherein the support material is a synthetic material selected from the group consisting of polypropylene, polyester, silicone, polyethylene, polyamide, titanium, stainless steel, polymethylmethacrylate, silk, cotton, polyglactic acid, polyglycolic acid, poliglecaprone, collagen, polydioxone, and polytetrafluoroethylene.

12. The composition of claim 1, further comprising an anti-adhesive.

13. The composition of claim 12, wherein the anti-adhesive is selected from the group consisting of heparin, streptokinase, urokinase, ancrod, and tissue plasminogen activator.

14. The composition of claim 1 further comprising an anti-inflammatory.

15. The composition of claim 14, wherein the anti-inflammatory is selected from the group consisting of steroids, non-steroidal anti-inflammatory agents, and chemotherapeutic agents.

16. The composition of claim 1 further comprising a growth factor.

17. The composition of claim 16, wherein the growth factor is selected from the group consisting of vascular endothelial growth factors, platelet-derived growth factors, epidermal growth factors, insulin-like growth factors, transforming growth factor beta, and fibroblast growth factor.

18. The composition of claim 1 further comprising a substance selected from the group consisting of antibiotics, antiviral agents, growth-inhibiting agents, antithrombotic agents, prothrombotic agents, immunosuppressive agents, angiogenic agents and anti-angiogenic agents.

19. The composition of claim 1, wherein the barrier material is attached to the support material using an adhesive.

20. The composition of claim 19, wherein the adhesive is selected from the group consisting of cyanoacrylate, glue, fibrin glue, fibrin, thrombin, plasma, platelet-poor plasma, platelet-rich plasma, polyactide, and cellular-derived hemostatic agents.

21. The composition of claim 1, wherein the barrier material is attached to the support material using a mechanical agent selected from the group consisting of sutures, staples, and lamination.

22. The composition of claim 1, wherein the support material is substantially encased by the barrier material.

23. A method of repairing a wound comprising:
(a) preparing the wound area;
(b) draping the wound with the composite of claim 1;
(c) attaching the composite to the wound area; and
(d) covering or closing the wound.

24. The method of claim 23 wherein the wound for repair is selected from the group consisting of pelvic defects, joint defects, abdominal defects, chest wall defects, cranial defects, hernias, congenital abnormalities, skin lesions, burns, surgical incisions, and traumatic wounds.

25. A composition for wound repair comprising a barrier material and a support material, wherein the barrier material is acellular dermal tissue.

26. The composition of claim 25, wherein the support material is polypropylene.

27. The composition of claim 25, further comprising an anti-adhesive.

28. The composition of claim 27, wherein the anti-adhesive is selected from the group consisting of heparin, streptokinase, urokinase, ancrod, and tissue plasminogen activator.

29. The composition of claim 25 further comprising an anti-inflammatory.

30. The composition of claim 29, wherein the anti-inflammatory is selected from the group consisting of steroids, non-steroidal anti-inflammatory agents, and chemotherapeutic agents.

31. The composition of claim 25 further comprising a growth factor.

32. The composition of claim 31, wherein the growth factor is selected from the group consisting of vascular endothelial growth factors, platelet-derived growth factors, epidermal growth factors, insulin-like growth factors, transforming growth factor beta, and fibroblast growth factor.

33. The composition of claim 25 further comprising a substance selected from the group consisting of antibiotics, antiviral agents, growth-inhibiting agents antithrombotic agents, prothrombotic agents, immunosuppressive agents, angiogenic agents and anti-angiogenic agents.

34. The composition of claim 25, wherein the barrier material is attached to the support material using an adhesive.

35. The composition of claim 34, wherein the adhesive is selected from the group consisting of cyanoacrylate, glue, fibrin glue, fibrin, thrombin, plasma, platelet-poor plasma, platelet-rich plasma, polyactide, and cellular-derived hemostatic agents.

36. The composition of claim 25, wherein the barrier material is attached to the support material using a mechanical agent selected from the group consisting of sutures, staples, and lamination.

37. The composition of claim 25, wherein the support material is substantially encased by the barrier material.

38. A method of repairing a wound comprising:
(a) preparing the wound area;
(b) draping the wound with the composite of claim 25;
(c) attaching the composite to the wound area; and
(d) covering or closing the wound.

39. The method of claim 38 wherein the wound for repair is selected from the group consisting of pelvic defects, joint defects, abdominal defects, chest wall defects, cranial defects, hernias, congenital abnormalities, skin lesions, burns, surgical incisions, and traumatic wounds.
